(19) United States
(12) Patent Application Publication
(10) Pub. No.: US 2011/0305747 A1
(43) Pub. Date: Dec. 15, 2011

(54) COMBINATION OF DAPSONE WITH OTHER ANTI-ACNE AGENTS

(75) Inventors: Gurpreet Ahluwalia, Orange, CA (US); Kevin S. Warner, Anaheim, CA (US); Haigang Chen, Petaluma, CA (US); Meidong Yang, Richmond, CA (US); Jeffrey R. Ehrhardt, San Clemente, CA (US); Frederick C. Beddingfield, Pacific Palisades, CA (US)

(73) Assignee: ALLERGAN, INC., Irvine, CA (US)

(21) Appl. No.: 13/013,511

(22) Filed: Jan. 25, 2011

Related U.S. Application Data
(63) Continuation-in-part of application No. 12/846,079, filed on Jul. 29, 2010.
(60) Provisional application No. 61/229,903, filed on Jul. 30, 2009.

(51) Int. Cl. A61K 9/127; A61P 29/00; A61P 17/00; A61P 17/02; A61K 31/192; A61P 17/10

(52) U.S. Cl. 424/450; 514/569

(57) ABSTRACT

A composition suitable for topical application that contains at least two active ingredients, one of these being dapsone and one selected from the group consisting of adapalene, tazarotene and treinion for the effective treatment of acne and other dermatological conditions.

### CROSS REFERENCE

This application is a continuation-in-part of U.S. patent application Ser. No. 12/846,079, filed on Jul. 29, 2010 and claims the priority thereto, the entire disclosure of which is incorporated herein by this specific reference.

### FIELD OF THE INVENTION

The present invention is directed to compositions and methods for the treatment of acne vulgaris and other dermatological conditions.

### BACKGROUND OF THE INVENTION

Acne is the most common skin disease that affects a large number of adolescents and young adults after they reach puberty. Though not a life threatening disease, it has serious psychological impact on the patient. Chronic inflammatory acne can also result in permanent scarring of the face.

There are multiple factors that contribute to the pathogenesis of acne, these include: 1. over activity of sebum production as a result of hormonal changes at puberty; 2. colonization of Propionibacterium acnes (P. acnes) in the pilosebaceous unit; 3. hyperkeratinization or abnormal desquamation of epithelium of the upper follicle (above the sebaceous gland) that results in blockage of the pilosebaceous canal; and, 4. formation of inflammatory molecules as a result of the action of P. acnes on sebaceous lipids.

The obstruction of the pilosebaceous canal and inflammation caused by P. acnes created inflammatory metabolites results in the formation of comedones. Excess sebum production as a result of hormonal changes at puberty, combined with increased epithelium turnover of the upper follicle leads to formation of microcomedones which progresses to inflammatory papules and pustules in acne. The combination of lipid rich sebum and protein rich desquamated cells provides an ideal environment for the growth and activity of P. acnes which converts the sebaceous lipids to the inflammatory free fatty acid molecules resulting in inflammatory acne lesions. The patient can have either non-inflammatory (open and closed comedones), inflammatory (papules and pustules) or a combination of both which most often is the case. Topical treatments are generally sufficient in most patients to control the acne lesions.

Because acne is a multifactorial condition, the marketed products work on one or more of the underlying factors contributing to acne for its treatment. There are number of prescription and over-the-counter (OTC) products available that treat acne; however, they all lack either desired efficacy or tolerability or both. Currently available products include antibiotics (topical and systemic), benzoyl peroxide, retinoids (topical and systemic), dapsone, and a number of other compounds.

The anti-acne molecule dapsone is marketed as a commercial product Aczone®. Aczone® is a 5% dapsone gel with a gritty texture due to insoluble particles of dapsone drugs. The insolubility of dapsone limits the bioavilability of dapsone upon application and its absorption through the skin and is therefore administered twice daily. At the biochemical and molecular level, dapsone exhibits an anti-inflammatory activity which provides a unique mechanism of action for this molecule in treatment of inflammatory acne lesions. However, its mechanism of action is not entirely understood. A complex combination of inflammatory pathways produce the clinical inflammation observed in acne. It is known that neutrophils significantly contribute to inflammatory acne. Dapsone is known to suppress neutrophil recruitment and local production of toxic products there by inhibiting neutrophil chemotaxis and reducing generation of oxygen free radicals. Dapsone further inhibits release of lysosomal enzymes and reduces and bocks inflammatory effects of prostaglandins & leukotrienes. These effects result in reduction of inflammatory acne lesions. In addition to its anti-inflammatory activity, dapsone is also effective against P. acnes. The MIC90 against P. acnes is 8 μg/ml.

Adapalene is a third generation retinoid, which are compounds related to Vitamin A, and has been approved by the FDA for the treatment of acne. Adapalene is known to moderate inflammatory processes but its mechanism of action is also not entirely understood. Adapalene products are sold with the concentrations of 0.1% and 0.3% w/v concentrations for gels and 0.1% w/v concentration for cream. Adapalene acts on retinoid receptors and appears to be a modifier of cellular differentiation, keratinization and inflammatory processes which are involved in the pathology of acne vulgaris. Absorption of adapalene from either 0.1% or 0.3% gel or cream is low. In one pharmacokinetic study, 16 patients suffering from acne vulgaris received 0.3% adapalene gel applied to the face, chest and back which is approximately a dosage of 2 mg/cm2. Fifteen patients resulted in quantifiable (LOQ=0.1 ng/mL) adapalene levels with a mean Cₘₐₓ of 0.553±0.466 ng/mL on Day 10 of treatment. Mean AUC₀₋₂₄ₕᵣ was 8.37±8.46 ng·h/mL as determined in 15 of the 16 patients on Day 10. Terminal apparent half-life, which was determined in 15 of 16 patients, ranged from 7 to 51 hours, with a mean of 17.2±10.2 hours. Adapalene was rapidly cleared from plasma and was not detected 72 hours after the last application for all but one subject.

### SUMMARY OF THE INVENTION

There is an unmet consumer need for an efficacious product for the treatment of acne vulgaris as the currently available products for treatment of acne vulgaris lack the desired efficacy and/or have side effects or tolerability issues that are undesired by the subjects.

A combination acne product would provide the benefit of enhanced efficacy compared to the products containing a single active agent by taking advantage of the synergistic mechanism of action of the active agents for treatment of acne. The present invention is directed to acne products with at least two active compounds and in particular are directed to dapsone and adapalene combination formulations for the use in the treatment of dermatological conditions such as acne vulgaris, rosacea, atopic dermatitis, treatment of chronic wounds, bed sores, keratosis piralis, psoriasis, cosmetic improvement of surgical and acne scars, sebaceous cysts, inflammatory dermatoses, post inflammatory hyperpigmentation, eczema, xerosis, pruritis, lichen planus, nodular prurigo, eczema, and miliaria and other dermatological conditions.

As used in the specification and claims, the term “about” refers to variations in the concentrations of active agents and excipients which would be considered by a regulatory authority, such as the FDA or EMEA, to be bioequivalent.

Some embodiments of the present invention include:

1) A dermatological composition comprising dapsone, adapalene, and water.

2) The dermatological composition of paragraph 1 wherein the composition comprises about 5% w/w dapsone, about 0.1% or about 0.3% w/w adapalene and is used for the treatment of acne vulgaris.

3) The dermatological composition of paragraph 2 wherein the composition is about 5% w/w dapsone and about 0.3% w/w adapalene.

4) The dermatological composition of paragraph 1 wherein the composition is a gel.

5) The compositions of paragraphs 1 and 4 wherein the composition is about 5% w/w dapsone, about 0.1% or 0.3% w/w adapalene, about 1.5% w/w benzyl alcohol, transcutol, about 5-25% w/w PEG 400, about 0.01% w/w EDTA, and about 0.03% w/w citric acid.

6) The compositions of paragraphs 1-5 wherein the composition further comprises hydroxyl ethyl cellulose at about 1-4% w/w.

7) The compositions of paragraphs 1-5 further comprising carbopol 980 at about 0.5-2% w/w.

8) The compositions of paragraphs 1-7 further comprising methyl paraben.

9) The compositions of paragraphs 1-8 further comprising lactic acid.

10) The compositions of paragraphs 1-9 further comprising glycerin.

11) The composition of paragraph 5 further comprising dimethyl isosorbide in 5-15% w/w.

12) The composition of paragraphs 1-5 wherein transcutol is present in the amount of about 25% w/w.

13) The compositions of paragraphs 1-12 wherein a buffer selected from the group consisting of NaOH, trolamine, and hydrochloric acid is added to adjust the pH.

14) The compositions of paragraphs 1-13 wherein the pH of the composition is about 5.5-6.0

15) The composition of paragraphs 1-5 further comprising about 2-3% hydroxyl ethyl cellulose.

16) The compositions of paragraphs 1-15 wherein the composition is in the form of one selected from the group consisting of a gel, emulsion, cream, liquid, paste, lotion, nanoemulsion, microemulsion, reverse emulsion and liposomal cream.

17) The compositions of paragraphs 1-16 wherein the composition may be used for treatment of one selected from the group consisting of acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, and miliaria and other dermatological conditions.

18) A method of treating acne vulgarus by application of one of the compositions of paragraphs 1-17.

19) The method of treatment of paragraph 17, wherein the application is applied once a day.

20) The method of treatment of paragraph 17, wherein the application is applied twice a day.

### BRIEF DESCRIPTION OF THE DRAWINGS

FIG. 1 is directed to dapsone and adapalene formulations for the treatment of dermatological conditions;

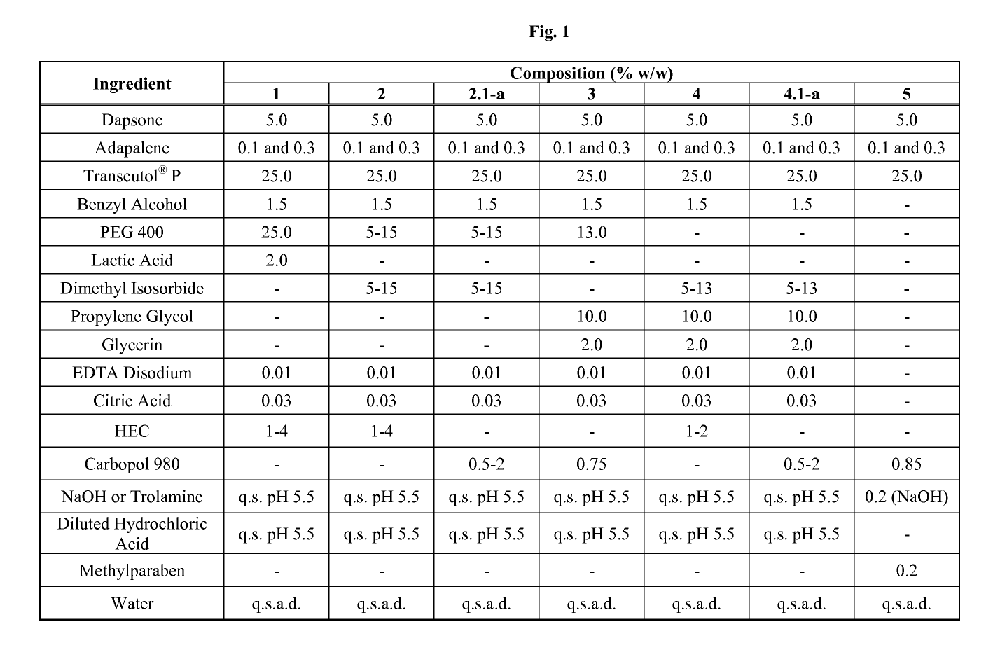

FIG. 2 is directed to variations of formulations for the treatment of dermatological conditions of Formula 1 of FIG. 1;

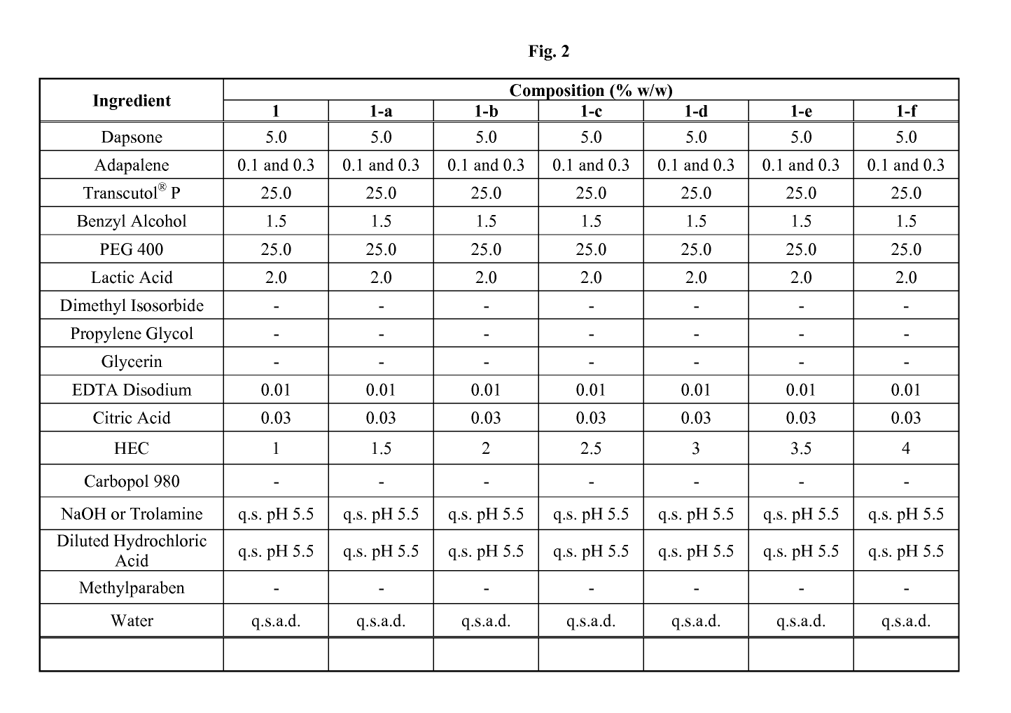

FIG. 3A is directed to variations of formulations for the treatment of dermatological conditions of Formula 2 of FIG. 1;

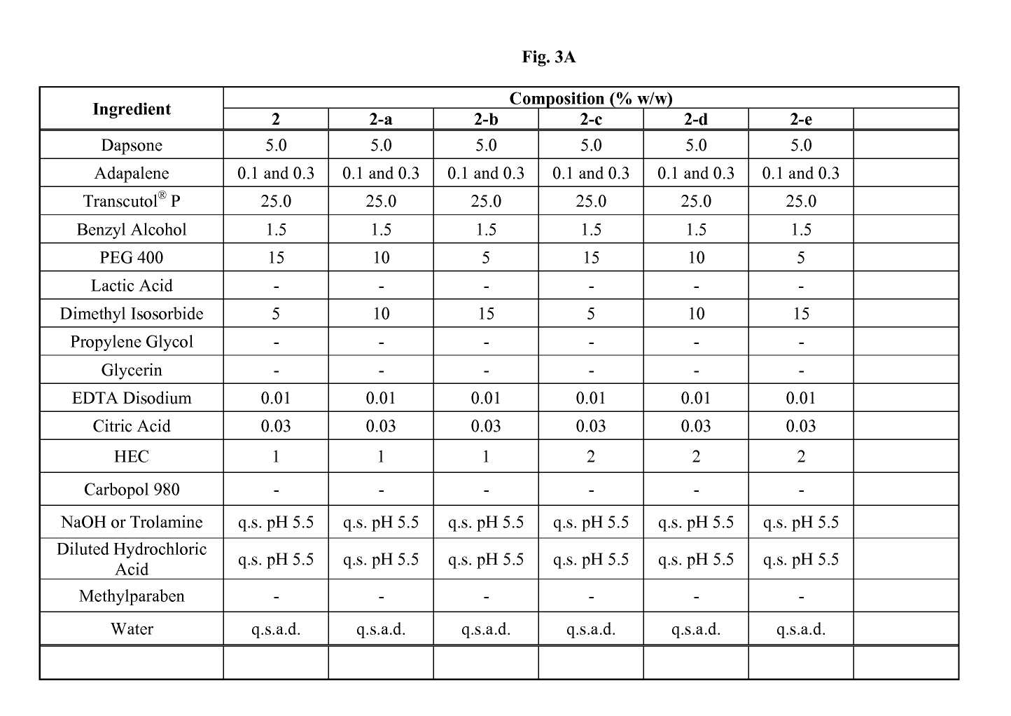

FIG. 3B is directed to variations of formulations for the treatment of dermatological conditions of Formula 2 of FIG. 1;

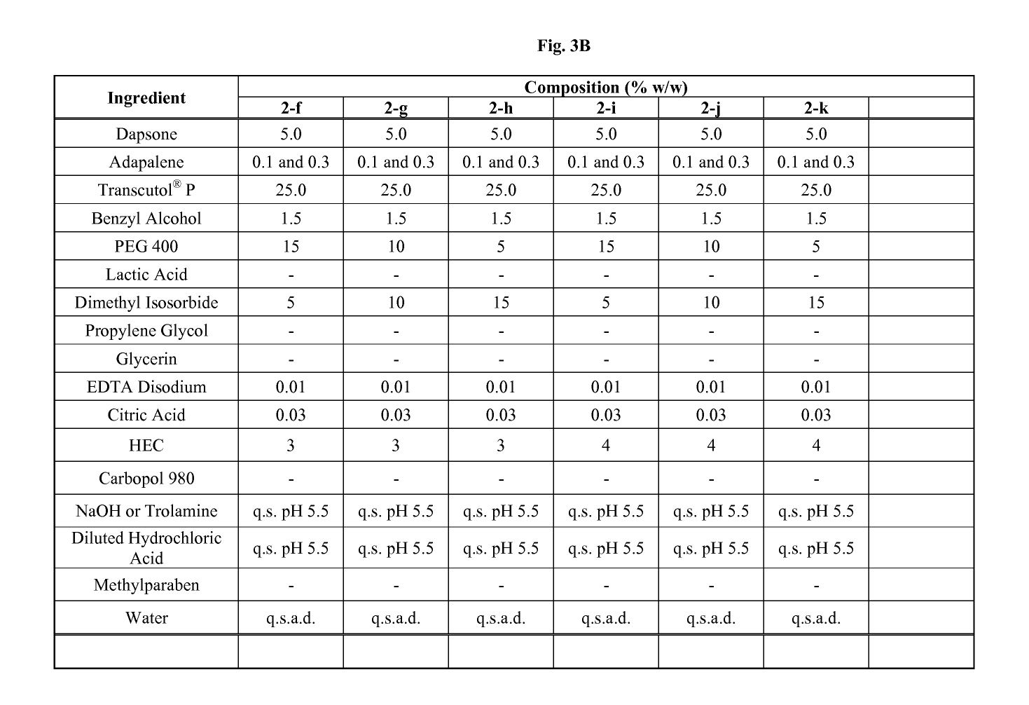

FIG. 3C is directed to variations of formulations for the treatment of dermatological conditions of Formula 2.1 of FIG. 1;

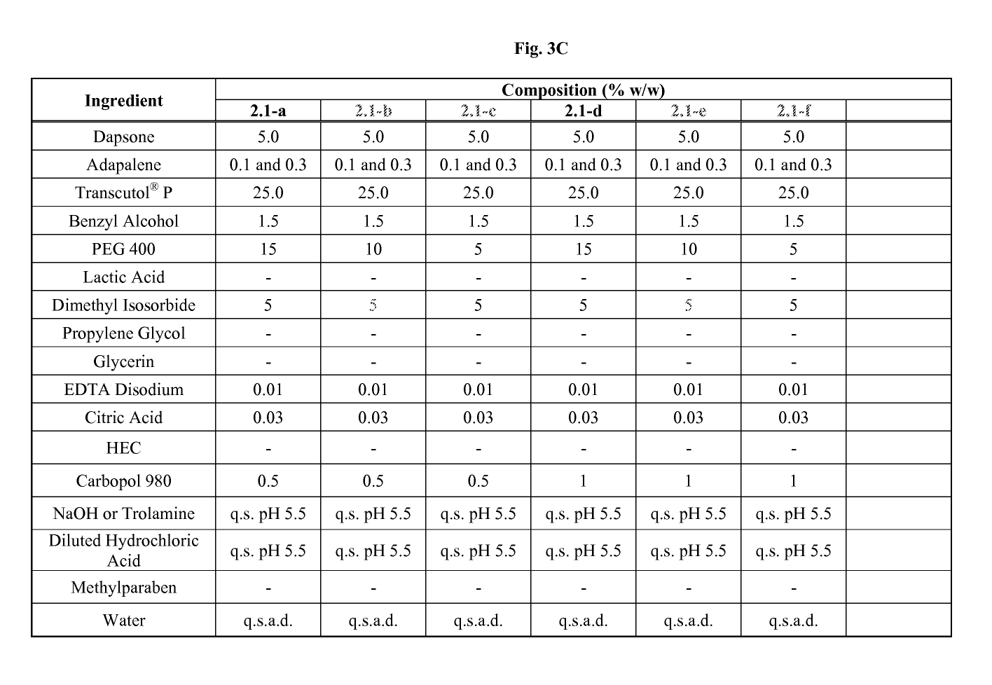

FIG. 3D is directed to variations of formulations for the treatment of dermatological conditions of Formula 2.1 of FIG. 1;

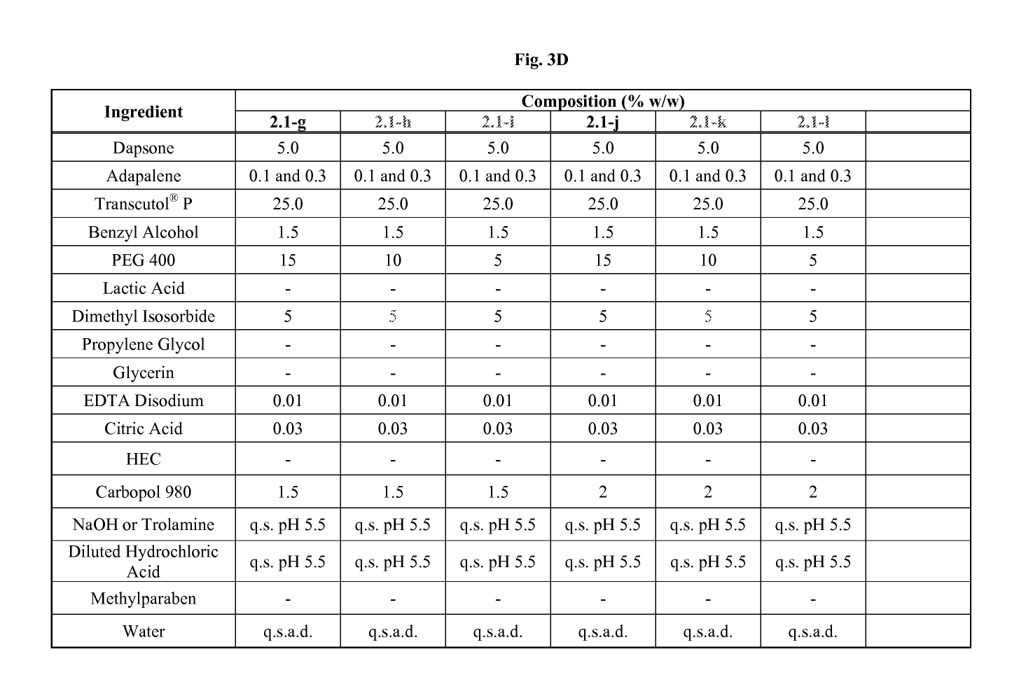

FIG. 4A is directed to variations of formulations for the treatment of dermatological conditions of Formula 4 of FIG. 1;

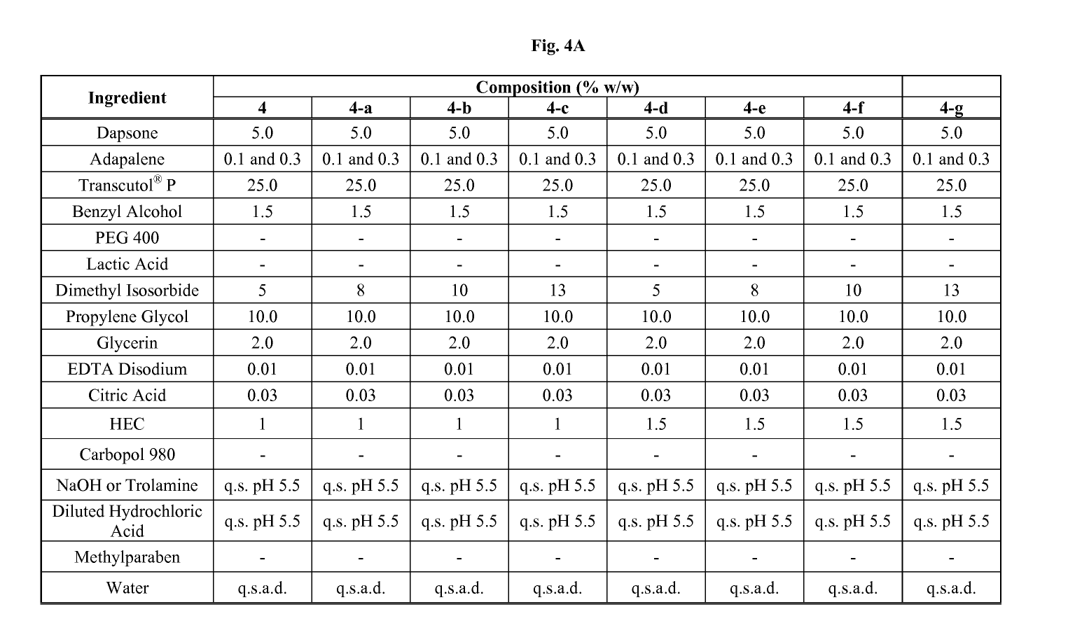

FIG. 4B is directed to variations of formulations for the treatment of dermatological conditions of Formula 4 of FIG. 1;

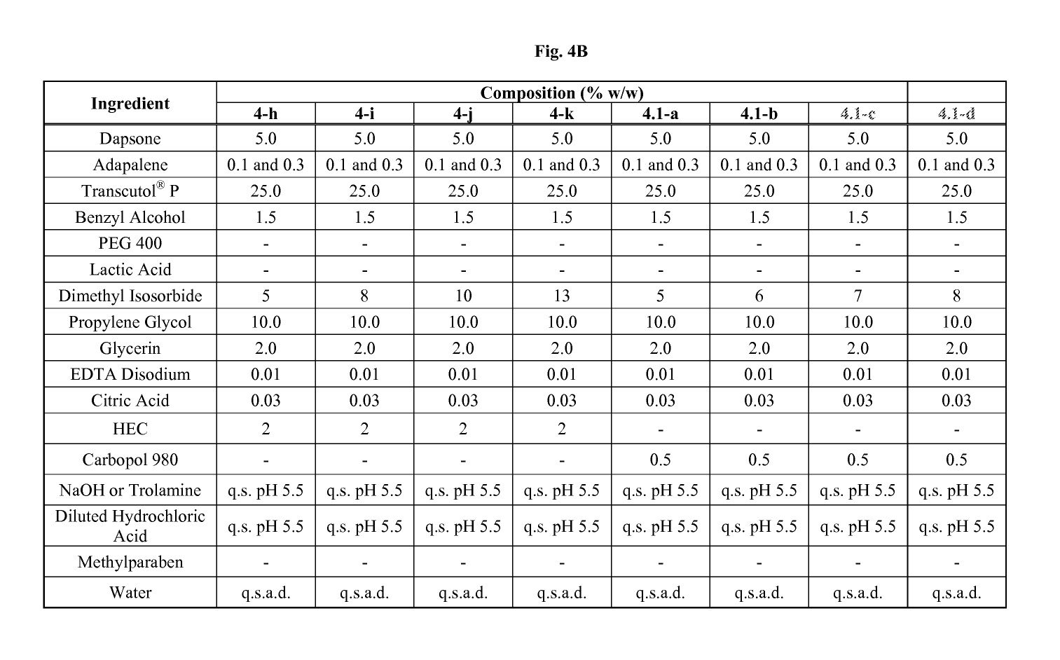

FIG. 4C is directed to variations of formulations for the treatment of dermatological conditions of Formula 4 of FIG. 1;

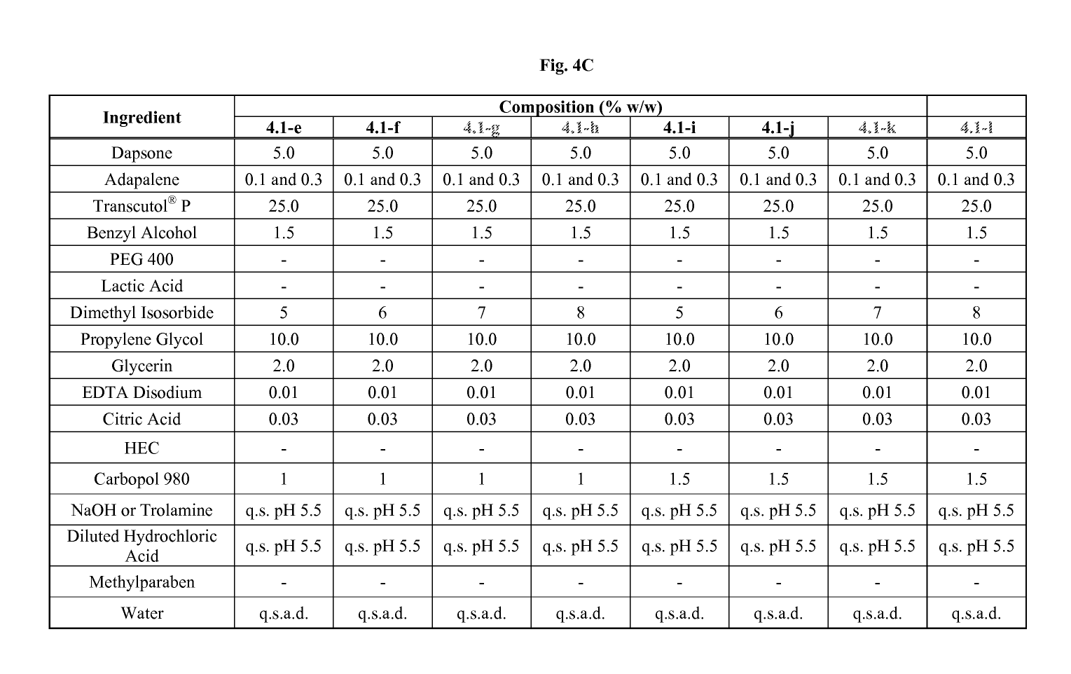

FIG. 4D is directed to variations of formulations for the treatment of dermatological conditions of Formula 4 of FIG. 1;

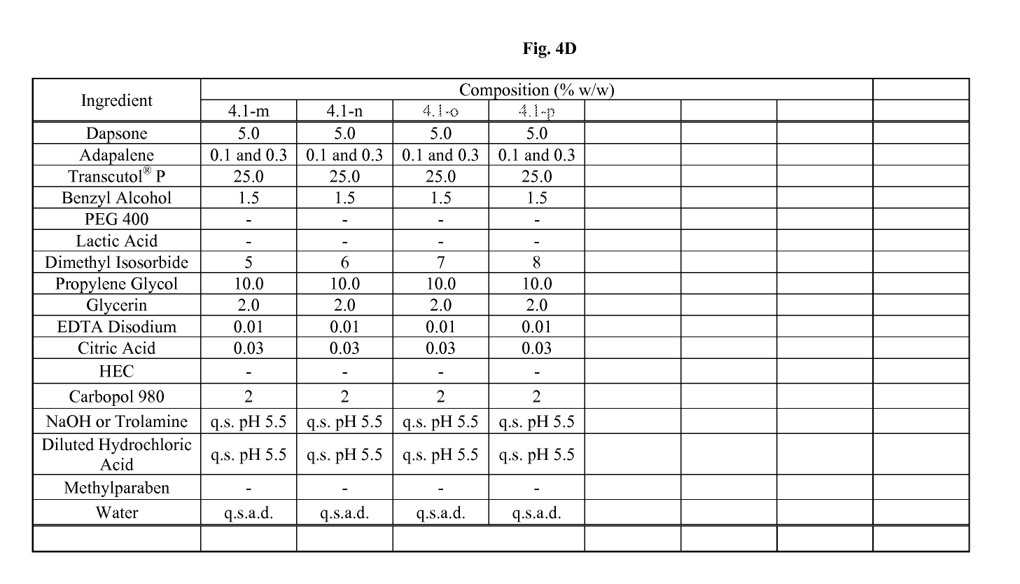

FIG. 5 is directed to dapsone and adapalene formulations for the treatment of dermatological conditions;

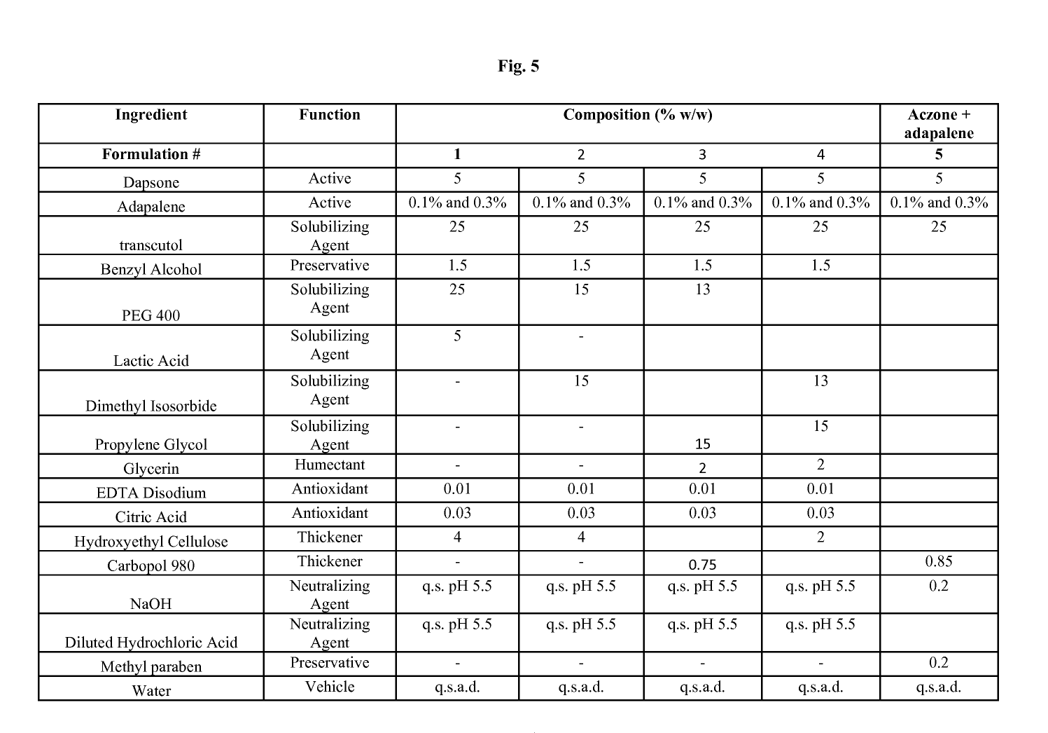

FIG. 6A is directed to dapsone and adapalene formulations for the treatment of dermatological conditions; and,

FIG. 6B is directed to dapsone and adapalene formulations for the treatment of dermatological conditions.

### DETAILED DESCRIPTION OF THE INVENTION

The present invention is directed to topical compositions for treatment of dermatological conditions which contain at least two active ingredients, one of these being dapsone and the other(s) selected from the list below for an effective treatment of acne and other dermatological conditions such as rosacea.

Some broad embodiments of the invention and possible combinations are found below:

Suitable compounds that can be combined with dapsone (2-10% w/w) include:
1. Agents with bactericidal and/or comedolytic properties:
   a. Benzoyl peroxide (2.5-10% w/w); and,
   b. other antimicrobial actives that are effective against P. acnes.
2. Agents that inhibit comedogenesis by reducing pilosebaceous canal obstruction or have keratolytic properties such as:
   a. Salicylic acid (0.5-3% w/w);
   b. Azelaic acid (up to 20% w/w);
   c. Sulfacetamide-sulfur (5-10% w/w); and,
   d. other keratolytic agents.
3. Agents that reduce sebaceous gland secretion and effect epithelial dysquamation:
   a. Retinoids:
      i. tretinoin or trans retinoic acid (0.02-0.1% w/w);
      ii. Tazarotene (0.05-0.1% w/w);
      iii. Adapalene (0.1-0.3% w/w); and,
      iv. additional retinoids.
4. Topical antibiotics for directly killing P. acnes:
   a. erythromycin (1-3% w/w);
   b. clindamycin (1-2% w/w); and,
   c. tetracycline (1-3% w/w).
      Potential combinations that can be used:

1. Dapsone (0.01%-10% w/w)+retinoid (0.001%-3% w/w)
Examples
   a. Dapsone 5% w/w+Adapalene 0.3% w/w;
   b. Dapsone 5% w/w+tazarotene 0.1% w/w; and,
   c. Dapsone 5% w/w+tretinoin 0.1% w/w.

2. Dapsone+benzoyl peroxide:
Examples
   a. Dapsone 5% w/w+benzoyl peroxide 5% w/w;

3. Dapsone+antibiotic:
Examples
   a. Dapsone 5% w/w+clindamycin 1% w/w.

4. Dapsone+keratolytic agent
Examples:
   a. Dapsone 5% w/w+Azelaic acid 20% w/w.

The concentration values (w/w) in parenthesis represent preferred concentration; however, other concentrations values (w/v) can be used dependent on the formulation characteristics and the desired level of efficacy and tolerability.

Dapsone possesses both anti-microbial and anti-inflammatory properties. It is the anti-inflammatory property of dapsone which is a key to its acne treatment. The anti-inflammatory activity of dapsone is attributed to its ability to suppress neutrophil recruitment and interference with chemotactic migration and local production of toxic products (Debol et al, 1997). Neutrophils contribute to the inflammation in all stages of acne. Inhibition of neutrophil myeloperoxidase and eosinophil peroxidase by dapsone suppress the production of hypochlorous acid that kills bacteria but also damages adjacent tissue (Kazmierowski et al, 1984). Dapsone also scavenges reactive oxygen species and minimizes inflammation associated with the generation of these highly reactive species (Maloff et al, 1988). Dapsone's antimicrobial activity is unrelated to its anti-inflammatory activity. Dapsone competitively inhibits dihydropteroate synthase, the bacterial enzyme responsible for the incorporation of para-aminobenzoic acid into dihydropteric acid, the immediate precursor of folic acid (Coleman, 1993). Microorganisms that need to synthesize their own folic acid are sensitive to this class of compounds (sulfones).

Adapalene, a synthetic analog of retinoic acid, selectively binds to retinoic acid receptor-beta, and the gamma nuclear receptors of retinoic acid. However, it does not bind to CRABPII (cellular retinol-binding protein II). Like retinoic acid, adapalene also activates nuclear receptors and inhibits transglutaminase I, an enzyme involved in the terminal differentiation of keratinocytes which may result in decreased microcomedone formation. The drug has also expressed comedolytic activity in the Rhino mouse containing a high density of spontaneous comedones (Allec et al, 1997). Based on their respective mechanisms of action, dapsone and adapalene provide a complementary anti-acne activity.

### Skin Penetration, and Efficacy and Safety of the Dapsone-Adapalene Combination

The efficacy and safety of a combination product is dependent on the formulation characteristics. Both dapsone and adapalene are difficult molecules to formulate in a dermatologically and aesthetically acceptable formulation that can deliver desired efficacy without compromising the safety and aesthetic of the final product. It is well recognized in the art that skin absorption is dependent on the physiochemical properties of the molecule and its interactions with the vehicle and the stratum cornium layer of skin, which is the principal barrier to penetration of molecules. For efficacy of acne products, there are two major routes of skin penetration: 1. diffusion through the stratum cornium barrier layer into the dermis and the general circulation, and 2. transport through the pilosebaceous canal (hair follicle opening) that leads directly to the sebaceous gland—the target for acne treatment. The pilosebaceous canal is filled with sebum, therefore for a molecule to effectively penetrate to the sebaceous gland it must first partition into the highly lipophylic sebum secretion.

Both dapsone and adapalene in their current marketed formulations, i.e., Aczone (5% dapsone) and Differin (0.1% or 0.3%), are present in the form of a suspension which is not ideal for achieving optimal delivery to the sebaceous glands either through skin diffusion or through pilosebaceous canal transport mechanism. This is likely to result in less than an optimal efficacy of the product.

### New Formulations for Dapsone and Adapalene Combination Product:

Unlike the marketed ACZONE® product where dapsone is a suspension and dapsone is in a partially soluble and insoluble state, dapsone is fully solubilized in formulations described in the present invention, thereby enhancing the probability for higher skin absorption of the molecule through stratum cornium diffusion. Additionally, the selected ratios of the vehicle components in the formulations of the present invention, aid in solubilizing sebum present in the pilosebaceous canal thereby increasing the partitioning of both dapsone and adapalene molecules in sebum and consequently their delivery to the targeted sebaceous gland. This targeted delivery approach is expected to result in higher accumulation of both dapsone and adapalene in the sebaceous gland and therefore a greater efficacy of the combination product, at the same time minimizing any dermal side effects from adapalene skin accumulation. The soluble dapsone in the formulations listed in the present invention can diffuse into the dermal layer providing an anti-inflammatory effect that is expected to further enhance the efficacy and safety of this combination product. To this end, animal studies conducted with these new formulations containing the combination of the two molecules show that the dermal tolerability is maintained. The new formulation were applied to the shaved back of rabbits and various dermal tolerability parameters, including irritation, erythema (redness), edema, dryness and scaliness were assessed. For controls, the marketed dapsone and adapalene formulations were used. The results showed comparable or better tolerability of the combination new products compared to the marketed products that contain the individual ingredients (dapsone or adapalene).

An additional disadvantage of the currently marketed product Aczone is the poor aesthetics related to ‘grittiness’ of the product feel that is contributed by the suspended dapsone. The new formulations where dapsone is fully solubilized eliminate this negative aesthetic component. Because of the difficulty of solubilizing dapsone in a vehicle that is not only aesthetically, but also dermatologically acceptable, the new formulations provided surprising stability for both dapsone and adapalene combination.

In a recent clinical trial, the safety and efficacy of dapsone gel co-administered with adapalene gel was assessed. The study design consisted of having patients apply the product Aczone® (5% w/w dapsone) twice a day, with morning and evening application. About 10 minutes after the evening application of Aczone®, patients applied a thin layer of 0.1% w/w adapalene gel. The 10 minute separation between applications of the two products ensured complete absorption of the Aczone® formulation into the skin to minimize the potential negative impact on adapalene or dapsone skin penetration. Application of the 0.1% w/w adapalene gel immediately after the Aczone® application may have resulted in a situation where the adapalene or dapsone would have a lower skin penetration because of the mixing of the two formulation vehicles. Further, the additional thickness of the combined formulation applications may increase the penetration distance of the two actives also resulting in reduced skin penetration of the actives.

The results of the trial showed that dapsone gel administered concurrently (but not together) with adapalene gel is safe and well tolerated for the treatment of acne vulgaris. One aspect of the present invention is a combination adapalene/dapsone topical formulation combining the two actives into one formulation. The novelty of this invention is in part attributable to the use of additional excipients (solubilizers) in combination with diethylene glycol monoethyl ether (“DGME”) in order to solubilize dapsone. Addition of cosolvents has enabled the complete dissolution of dapsone in the formulation and an increase in the solubility of adapalene (adapalene is not completely solubilized in these formulations). The increased concentration of dissolved dapsone and adapalene versus the marketed product comparators administered concurrently will increase the rate of skin penetration of both drugs into and through the skin

Topical dosage forms of the present invention include, but are not limited to, solutions, gels, creams, ointments, foams, emulsions, films, and facial/skin peels. The present invention is directed to topical dapsone and adapalene formulations which are formulated to optimize the dermal delivery profile of adapalene and dapsone to effectively treat acne and other dermatological conditions and improve the efficiency of pharmaceutical products applied to the skin.

Examples of some formulations encompassed by the present invention excipients and concentration ranges are summarized in Table 1 below:

TABLE 1
Example Excipient Composition Ranges Utilized in Adapalene/Dapsone
Topical Formulations:

| Ingredient | Function | Composition (% w/w) |
|---|---|---|
| Dapsone | Active | 0.5-10 |
| Adapalene | Active | 0.1-0.3 |
| Carbomer 980 | Thickener | 0.05-1.5 |
| Hydroxyethyl cellulose | | 1-8% |
| Hydroxypropyl cellulose | | 1-6% |
| NaOH | Neutralizing Agent | 0.01-2.0 |
| Trolamine | Neutralizing Agent | 0.01-2.0 |
| Ethanol | Solubilizers | 1-90 |
| Lactic acid | | 1-10 |
| diethylene glycol | | 1-50 |
| monoethyl ether | | |
| propylene glycol | | 1-60 |
| Dimethyl isosorbide | | 1-30 |
| Polyethylene glycol 400 | | 1-50 |
| Hexylene glycol | | 1-50 |
| Isostearyl alcohol | | 0.5-10 |
| Medium chain triglycerides | | 0.5-10 |
| Isopropyl myristate | | 2-10 |
| Benzyl alcohol | Preservative | 0.5-5 |
| Methyl Paraben | Preservative | 0.1-0.3 |
| Propyl Paraben | Preservative | 0.01-1 |
| Benzalkonium Chloride | Preservative | 0.1-0.2 |
| Sorbic Acid | Preservative | 0.1-2.7 |
| Glycerol | Humectant | 1-20 |
| Polyvinyl alcohol | Film forming | 1-30 |
| Water | Vehicle | 1-90 |
| EDTA Disodium | Antioxidant | 0.005-0.02 |
| Citric Acid | Antioxidant | 0.015-0.06 |
| Butylated hydroxytoluene | Antioxidant | 0.005-1 |
| Butylated hydroxyanisole | Antioxidant | 0.01-0.25 |
| Propyl gallate | Antioxidant | 0.01-0.1 |
| Elastomer 10 | Thickener | 0.1-90 |
| ST Wax 30 | Thickener | 0.1-50 |
| Dimethiconol blend 20 | Thickener | 0.1-50 |
| Emulsifier 10 | Emulsifier | 0.1-50 |
| cyclomethicone 5 | Solvent | 0.1-50 |
| Silicone fluid | Solvent | 0.1-50 |
| Silky wax 10 | Thickener | 0.1-50 |

Further specific compositions of the present invention of 5% w/w dapsone and 0.1% w/w and 0.3% w/w adapalene formulations include but are not limited to:

TABLE 2A
Adapalene/Dapsone Topical Formulations

| Ingredient | Function | Composition (% w/w) | | | | | | | | |
|---|---|---|---|---|---|---|---|---|---|---|
| Dapsone | Active | 5 | 5 | 5 | 5 | 5 | 5 | 5 | 5 | 5 |
| Adapalene | Active | 0.1% | 0.1% | 0.1% | 0.1% | 0.1% | 0.1% | 0.1% | 0.1% | 0.1% |
| | | or | or | or | or | or | or | or | or | or |
| | | 0.3% | 0.3% | 0.3% | 0.3% | 0.3% | 0.3% | 0.3% | 0.3% | 0.3% |
| diethylene | Solubilizing | 25 | 20 | 25 | 20 | 25 | 25 | 25 | 25 | 25 |
| glycol | Agent | | | | | | | | | |
| monoethyl | | | | | | | | | | |
| ether | | | | | | | | | | |
| Benzyl | Preservative | 1.5 | 1.5 | 1.5 | 1.5 | 1.5 | 1.5 | 1.5 | 1.5 | — |
| Alcohol | | | | | | | | | | |
| PEG 400 | Solubilizing | 25 | 20 | 25 | 20 | 15 | — | — | — | — |
| | Agent | | | | | | | | | |
| Lactic Acid | Solubilizing | 5 | 4 | — | — | — | — | — | — | — |
| | Agent | | | | | | | | | |
| Dimethyl | Solubilizing | — | — | — | — | 15 | — | — | — | — |
| Isosorbide | Agent | | | | | | | | | |
| Propylene | Solubilizing | — | — | — | — | — | 20 | 20 | 10 | — |
| Glycol | Agent | | | | | | | | | |
| Glycerin | Humectant | — | — | — | — | — | 10 | 10 | 2 | — |
| Isopropyl | Solubilizing | — | — | — | — | — | — | — | — | 5 |
| Myristate | Agent | | | | | | | | | |
| EDTA | Antioxidant | 0.01 | 0.01 | 0.01 | 0.01 | 0.01 | 0.01 | 0.01 | 0.01 | — |
| Disodium | | | | | | | | | | |
| Citric Acid | Antioxidant | 0.03 | 0.03 | 0.03 | 0.03 | 0.03 | 0.03 | 0.03 | 0.03 | — |
| Hydroxyethyl | Thickener | 4 | 3 | — | — | 4 | — | — | — | — |
| Cellulose | | | | | | | | | | |
| Carbopol 980 | Thickener | — | — | — | 0.75 | — | 0.75 | 0.75 | 0.75 | — |
| Hydroxypropyl | Thickener | — | — | — | — | — | — | — | — | 3 |
| Cellulose | | | | | | | | | | |
| NaOH | Neutralizing | 1.5 | 1.2 | q.s. | q.s. | q.s. | q.s. | q.s. | q.s. | — |
| | Agent | | | pH 5.5 | pH 5.5 | pH 5.5 | pH 5.5 | pH 5.5 | pH 5.5 | |
| Diluted | Neutralizing | — | — | q.s. | q.s. | q.s. | q.s. | q.s. | q.s. | — |
| Hydrochloric | Agent | | | pH 5.5 | pH 5.5 | pH 5.5 | pH 5.5 | pH 5.5 | pH 5.5 | |
| Acid | | | | | | | | | | |
| Ethanol | Solubilizer | — | — | — | — | — | — | — | — | 60 |
| Water | Vehicle | q.s.a.d. | q.s.a.d. | q.s.a.d. | q.s.a.d. | q.s.a.d. | q.s.a.d. | q.s.a.d. | q.s.a.d. | — |

TABLE 2B
Adapalene/Dapsone Topical Formulations (cont.)

| Ingredient | Function | Composition (% w/w) | | |
|---|---|---|---|---|
| Dapsone | Active | 5 | 5 | 5 |
| Adapalene | Active | 0.1% or | 0.1% or | 0.1% or |
| | | 0.3% | 0.3% | 0.3% |
| diethylene | Solubilizing | 25 | 25 | 25 |
| glycol | Agent | | | |
| monoethyl | | | | |
| ether | | | | |
| Benzyl Alcohol | Preservative | 1.5 | 1.5 | 1.5 |
| PEG 400 | Solubilizing | 13 | — | — |
| | Agent | | | |
| Dimethyl | Solubilizing | — | 13 | 13 |
| Isosorbide | Agent | | | |
| Propylene | Solubilizing | 15 | 15 | 15 |
| Glycol | Agent | | | |
| Glycerin | Humectant | 2 | 2 | 2 |
| EDTA | Antioxidant | 0.01 | 0.01 | 0.01 |
| Disodium | | | | |
| Citric Acid | Antioxidant | 0.03 | 0.03 | 0.03 |
| Hydroxyethyl | Thickener | — | 2 | — |
| Cellulose | | | | |
| Carbopol 980 | Thickener | 0.75 | — | — |
| Hydroxypropyl | Thickener | — | — | 2 |
| Cellulose | | | | |
| NaOH | Neutralizing | q.s. pH 5.5 | q.s. pH 5.5 | q.s. pH 5.5 |
| | Agent | | | |
| Diluted | Neutralizing | q.s. pH 5.5 | q.s. pH 5.5 | q.s. pH 5.5 |
| Hydrochloric | Agent | | | |
| Acid | | | | |
| Water | Vehicle | q.s.a.d. | q.s.a.d. | q.s.a.d. |

The formulations of the present invention can be made as follows based on the excipients:

Process for Making Lactic Acid Containing Formulations:

The combination adapalene/dapsone gels were prepared as follows:
a. Weigh the Transcutol into a kettle. Add the dapsone, lactic acid, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

### Process for Making DMI/Hydroxyethyl Cellulose Containing Formulations:

The combination adapalene/dapsone gels were prepared as follows:
a. Weigh the Transcutol into a kettle. Add the dapsone, dimethyl isosorbide, polyethylene glycol 400, benzyl alcohol. Stir with propeller mixer at room temperature. Mix until dissolved;
b. Add water, EDTA, and citric acid to mixture in step a. Mix until dissolved.
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

### Process for Making DMI/Carbopol Containing Formulations:

The combination adapalene/dapsone gels were prepared as follows:
a. Weigh the Transcutol into a kettle. Add the dapsone, dimethyl isosorbide, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

### Process for Making PG/PEG Containing Formulations:

The combination adapalene/dapsone gels were prepared as follows:
a. Weigh the Transcutol into a kettle. Add the dapsone, propylene glycol, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

### Process for Making PG/DMI/Carbopol Containing Formulations:

The combination adapalene/dapsone gels were prepared as follows:
a. Weigh the Transcutol into a kettle. Add the dapsone, propylene glycol, dimethyl isosorbide, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

### Process for Making PG/DMI/HEC Containing Formulations:

The combination adapalene/dapsone gels were prepared as follows:
a. Weigh the Transcutol into a kettle. Add the dapsone, propylene glycol, dimethyl isosorbide, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

The most effective dapsone and adapalene composition is selected based on clinical studies. For example, a clinical study is conducted by forming two treatment groups, one with daily application of a selected dapsone and adapalene formulation, and twice daily topical application of the same selected dapsone and adapalene formulation to the acne area of the skin for a period of 12 weeks. Two control groups are formed with application once and twice daily of a vehicle consisting of the same excipients but no active ingredients. The patient's inflammatory and non-inflammatory acne lesion counts should be recorded at baseline before initiation of treatment and then at select intervals throughout the study. The reduction in total, non-inflammatory or inflammatory lesions counts provides determination of the efficacy of the formulations. The established Global Acne Assessment Score (GAAS) should be used to assess efficacy of the product. The tolerability of the product can be determined by assessment of skin dryness, irritation, sensitivity and redness as a result of treatment. A product is considered to have better tolerability if there is less effect on these parameters.

### Application Method:

1. A suitable application method is topical cream, gel, lotion, ointment, foam, liquid or a semi solid preparation. A topical preparation may contain additional ingredients to provide aesthetic and moisturizing and anti-inflammatory benefits to the skin. Generally,
   a. A gel or liquid preparation can be alcohol or aqueous based or a combination of two;
   b. A nanoemulsion or microemulsion preparation can be used for enhanced delivery of actives;
   c. A liposomal cream or lotion preparation can be used for enhanced delivery of actives; and
   d. A foam preparation can be a quick breaking foam with additional emollient components.
2. Topical preparations that result in slow release or controlled release of the active agent can also be used to provide an optimal efficacy and tolerability balance.
3. Active ingredients encapsulated in micro beads or adsorbed on microsponge can be used for control release and in addition solve any incompatibility issues between the formulation ingredients.
4. The application is preferably once a day or more frequent depending on the desired effect.

Dapsone 5%/Adapalene 0.1% Gels

Formulation #

| Ingredient | 1 | 2 | 3 | 4 | 5 |
|---|---|---|---|---|---|
| Dapsone | 5 | 5 | 5 | 5 | 5 |
| Adapalene | 0.1 | 0.1 | 0.1 | 0.1 | 0.1 |
| Transcutol ® P | 25 | 25 | 25 | 25 | 25 |
| Benzyl Alcohol | 1.5 | 1.5 | 1.5 | 1.5 | — |
| PEG 400 | 20 | 13 | 13 | — | — |
| (super refined) | | | | | |
| Dimethyl Isosorbide | — | 5 | — | 11 | — |
| (super refined) | | | | | |
| Propylene Glycol | 5 | — | 10 | 10 | — |
| Glycerin | | — | — | 2 | — |
| EDTA | 0.01 | 0.01 | 0.01 | 0.01 | — |
| Citric Acid | 0.03 | 0.03 | 0.03 | 0.03 | — |
| Carbopol 980 | 1.0 | 1.5 | 1.0 | 1.5 | 0.85 |
| Trolamine | q.s. pH | q.s. pH | q.s. pH | q.s. pH 5.5-6.0 | — |
| | 5.5-6.0 | 5.5-6.0 | 5.5-6.0 | | |
| NaOH | — | — | — | — | 0.2 |
| Diluted | q.s. pH | q.s. pH | q.s. pH | q.s. pH 5.5-6.0 | — |
| Hydrochloric | 5.5-6.0 | 5.5-6.0 | 5.5-6.0 | | |
| Acid | | | | | |
| Methylparaben | — | — | — | — | 0.2 |
| Water | q.s. | q.s. | q.s. | q.s. | q.s. |

### Application of the Formulations of the Present Invention:

### Example #1

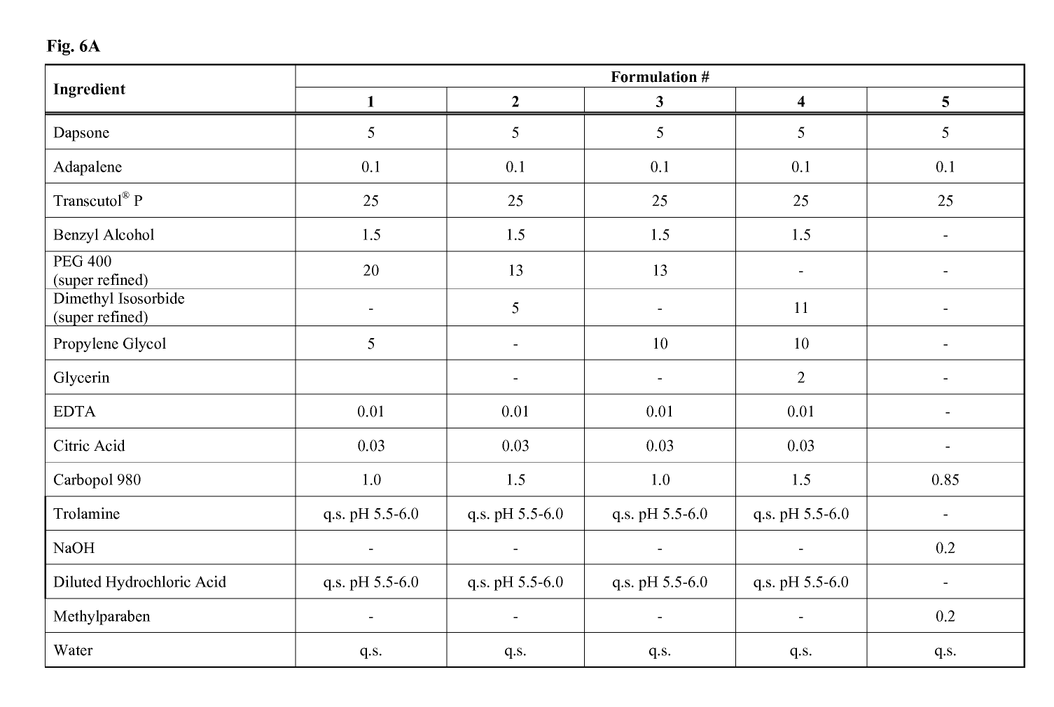

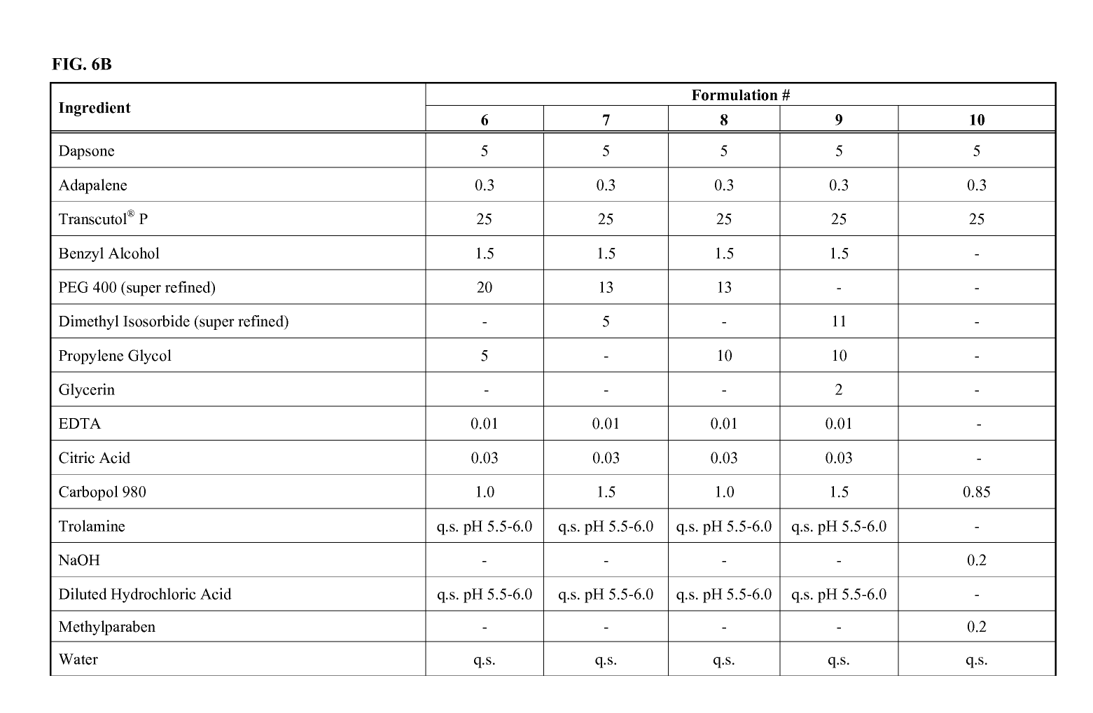

### Application of 0.3% w/w Adapalene of Formula 6 in FIG. 6B

A 17 year old Caucasian male patient suffers acne vulgaris with a combination of inflammatory and non-inflammatory lesions and applies a 0.3% w/w adapalene formulation according to formulation #6 in FIG. 6B. The 17 year old male patient applies the 0.3% w/w adapalene composition of Formula 6 once daily for 12 weeks. After 12 weeks, the 17 year old male patient experiences a 32% reduction in inflammatory and non-inflammatory lesions.

### Example #2

### Application of 0.3% w/w Adapalene of Formula 8 in FIG. 6B

A 16 year old Caucasian female patient suffers acne vulgaris with a combination of inflammatory and non-inflammatory lesions and applies a 0.3% w/w adapalene formulation according to formulation #8 in FIG. 6B. The 16 year old female patient applies the 0.3% w/w adapalene composition of Formula 8 once daily for 12 weeks. After 12 weeks, the 16 year old female patient experiences a 41% reduction in inflammatory and non-inflammatory lesions.

### Example #3

### Application of 0.1% w/w Adapalene of Formula 2 in FIG. 6A

A 23 year old African-American female patient suffers acne vulgaris with a combination of inflammatory and non-inflammatory lesions and applies a 0.1% w/w adapalene formulation according to formulation #2 in FIG. 6A. The 23 year old female patient applies the 0.1% w/w adapalene composition of Formula 2 once daily for 12 weeks. After 12 weeks, the 23 year old female patient experiences a 24% reduction in inflammatory and non-inflammatory lesions.

### Example #4

### Application of 0.3% w/w Adapalene of Formula 7 in FIG. 6B

A 19 year old Caucasian female patient suffers acne vulgaris with a combination of inflammatory and non-inflammatory lesions and applies a 0.3% w/w adapalene formulation according to formulation #7 in FIG. 6B. The 19 year old female patient applies the 0.3% w/w adapalene composition of Formula 7 once daily for 12 weeks. After 12 weeks, the patient experiences a 248% reduction in inflammatory and non-inflammatory lesions.

### Example #5

### Application of 0.1% w/w Adapalene of Formula 3 in FIG. 6A

An 18 year old African-American male patient suffers acne vulgaris with a combination of inflammatory and non-inflammatory lesions and applies a 0.1% w/w adapalene formulation according to formulation #3 in FIG. 6A. The 18 year old male patient applies the 0.1% w/w adapalene composition once daily for 12 weeks. After 12 weeks, the 18 year old male patient experiences a 29% reduction in inflammatory and non-inflammatory lesions.

### Example #6

### Application of 0.3% w/w Adapalene of Formula 9 in FIG. 6B

An 23 year old Asian female patient suffers acne vulgaris with a combination of inflammatory and non-inflammatory lesions and applies a 0.3% w/w adapalene formulation according to formulation #9 in FIG. 6B. The 23 year old patient applies the 0.3% w/w adapalene composition once daily for 12 weeks. After 12 weeks, the patient experiences a 25% reduction in inflammatory and non-inflammatory lesions.

### Example #7

### Application of 0.1% w/w Adapalene of Formula 4 in FIG. 6A

An 18 year old African-American male patient suffers acne vulgaris with a combination of inflammatory and non-inflammatory lesions and applies a 0.1% w/w adapalene formulation according to formulation #4 in FIG. 6A. The 18 year old male patient applies the 0.1% w/w adapalene composition once daily for 12 weeks. After 12 weeks, the 18 year old male patient experiences a 29% reduction in inflammatory and non-inflammatory lesions.

### Example #8

### Application of 0.3% w/w Adapalene of Formula 10 in FIG. 6B

A 17 year old Caucasian female patient suffers acne vulgaris with a combination of inflammatory and non-inflammatory lesions and applies a 0.3% w/w adapalene formulation according to formulation #10 in FIG. 6B. The 17 year old male patient applies the 0.3% w/w adapalene composition twice daily for 12 weeks. After 12 weeks, the 17 year old male patient experiences a 41% reduction in inflammatory and non-inflammatory lesions.

### Example #9

### Application of 0.1% w/w Adapalene of Formula 5 in FIG. 6A

A 16 year old Caucasian female patient suffers acne vulgaris with a combination of inflammatory and non-inflammatory lesions and applies a 0.1% w/w adapalene formulation according to formulation #5 in FIG. 6A. The 16 year old female patient applies the 0.1% w/w adapalene composition once daily for 12 weeks. After 12 weeks, the patient experiences a 27% reduction in inflammatory and non-inflammatory lesions.

### Example #10

### Application of 0.3% w/w Adapalene of Formula 10 in FIG. 6B

A 19 year old Caucasian female patient suffers acne vulgaris with a combination of inflammatory and non-inflammatory lesions and applies a 0.3% w/w adapalene formulation according to formulation #10 in FIG. 6B. The 19 year old female patient applies the 0.3% w/w adapalene composition twice daily for 12 weeks. After 12 weeks, the patient experiences a 38% reduction in inflammatory and non-inflammatory lesions.

### Example #11

### Application of 0.3% w/w Adapalene of Formula 6 in FIG. 6B

A 37 year old Caucasian male patient suffers from rosacea and applies a 0.3% w/w adapalene formulation according to formulation #6 in FIG. 6B. The 37 year old male patient applies the 0.3% w/w adapalene composition of Formula 6 once daily for 12 weeks. After 12 weeks, the 37 year old male patient experiences a reduction in the symptoms of rosacea.

### Example #12

### Application of 0.3% w/w Adapalene of Formula 8 in FIG. 6B

A 37 year old Caucasian male patient suffers from rosacea and applies a 0.3% w/w adapalene formulation according to formulation #8 in FIG. 6B. The 37 year old male patient applies the 0.3% w/w adapalene composition of Formula 8 once daily for 12 weeks. After 12 weeks, the 37 year old male patient experiences a reduction in the symptoms of rosacea.

1) A dermatological composition comprising dapsone, adapalene and water.

2) The dermatological composition of claim 1 wherein the composition comprises about 5% w/w dapsone and about 0.1% w/w adapalene and is used for the treatment of acne vulgaris.

3) The dermatological composition of claim 2 wherein the composition is about 5% w/w dapsone and about 0.3% w/w adapalene.

4) The dermatological composition of claim 1 wherein the composition is a gel.

5) The composition of claim 1 wherein the composition is about 5% w/w dapsone, about 0.3% w/w adapalene, about 1.5% w/w benzyl alcohol, transcutol, about 5-25% w/w PEG 400, about 0.01% w/w EDTA and about 0.03% w/w citric acid.

6) The composition of claim 5 wherein the composition further comprises a buffer selected from the group consisting of trolamine and hydrochloric acid.

7) The composition of claim 5 further comprising carbopol 980 at about 0.5-2% w/w.

8) The composition of claim 5 further comprising methyl paraben.

9) The composition of claim 5 further comprising lactic acid.

10) The composition of claim 5 further comprising glycerin.

11) The composition of claim 5 further comprising dimethyl isosorbide at about 5-15% w/w.

12) The composition of claim 5 wherein transcutol is present in the amount of about 25% w/w.

13) The composition of claim 5 wherein a buffer selected from the group consisting of NaOH, trolamine, and hydrochloric acid is added to adjust the pH.

14) The composition of claim 13 wherein the pH of the composition is about 5.5-6.0

15) The composition of claim 5 further comprising about 2-3% hydroxyl ethyl cellulose.

16) The composition of claim 1 wherein the composition is in the form of one selected from the group consisting of a gel, emulsion, cream, liquid, paste, lotion, nanoemulsion, microemulsion, reverse emulsion and liposomal cream.

17) The composition of claim 5 wherein the composition may be used for treatment of one condition selected from the group consisting of acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, and miliaria and other dermatological conditions.

18) A method of treating acne vulgarus by application of the composition comprising 5% w/w dapsone, about 0.3% w/w adapalene, about 1.5% w/w benzyl alcohol, transcutol, about 5-25% w/w PEG 400, about 0.01% w/w EDTA and about 0.03% w/w citric acid.

19) The method of treatment of claim 18, wherein the application is once a day.

20) The method of treatment of claim 18, wherein the application is twice a day.
